SEQUENCE LISTING (3 sequences)

SEQ ID NO: 1 - protein, 214 aa, Artificial Sequence
  Synthetic Light Chain of polyvalent bispecific antibody P4-G1-M1.3 (MM-141)
DIQMTQSPSSLSASLGDRVTITCRASQGISSYLAWYQQKPGKAPKLLIYAKSTLQSGVPSRFSGSGSGTDFTLTISSLQPEDSATYYCQQYWTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 720 aa, Artificial Sequence
  Synthetic Heavy Chain of polyvalent bispecific antibody P4-G1-M1.3 (MM-141)
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWDSGSTGYADSVKGRFTISRDNAKNSLYLQMNSLRAEDTALYYCARDLGAYQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSPGNQWVFGGGTKVTVLG

SEQ ID NO: 3 - protein, 246 aa, Artificial Sequence
  Synthetic linker
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

(19) United States
(12) Patent Application Publication
(10) Pub. No.: US 2017/0233491 A1
(43) Pub. Date: Aug. 17, 2017

(54) DOSAGE AND ADMINISTRATION OF COMBINATION THERAPIES COMPRISING ISTIRATUMAB, USES AND METHODS OF TREATMENT

(71) Applicant: Merrimack Pharmaceuticals, Inc., Cambridge, MA (US)

(72) Inventors: Alexey Alexandrovich LUGOVSKOY, Belmont, MA (US); Jason BAUM, Needham, MA (US); Chrystal U. LOUIS, Somerville, MA (US)

(73) Assignee: Merrimack Pharmaceuticals, Inc., Cambridge, MA (US)

(21) Appl. No.: 15/410,381

(22) Filed: Jan. 19, 2017

Related U.S. Application Data
(60) Provisional application No. 62/332,991, filed on May 6, 2016.
(60) Provisional application No. 62/286,146, filed on Jan. 22, 2016.
(60) Provisional application No. 62/280,606, filed on Jan. 19, 2016.

(51) Int. Cl. C07K 16/32; A61K 9/00; A61K 31/7068; A61K 31/337; A61K 39/395; C07K 16/28

(52) U.S. Cl. CPC C07K 16/32; A61K 39/39558; C07K 16/2863; A61K 31/7068; A61K 31/337; A61K 47/48284; A61K 47/48892; A61K 9/0019; C07K 2317/31; A61K 2039/505

(57) ABSTRACT

Disclosed herein are methods of treating pancreatic cancer in a human patient by co-administering istiratumab in combination with gemcitabine and nab-paclitaxel, and dosage regimens for the same.

### RELATED APPLICATIONS

This application claims priority to U.S. Provisional Application No. 62/280,606, filed on Jan. 19, 2016; 62/286,146, filed on Jan. 22, 2016; and 62/332,991, filed on May 6, 2016, respectively. The contents of the aforementioned applications are hereby incorporated by reference.

### FIELD

Provided are methods of treating a patient with cancer with a targeted therapy in combination with chemotherapies. Additionally, methods of determining whether the patient is likely to respond to a treatment with the aforementioned combinations are described.

### BACKGROUND

Despite recent advances in the treatment of pancreatic cancer (e.g., metastatic pancreatic cancer (MPC)), overall prognosis for patients remains poor. This may be attributed in part to the ability of pancreatic tumors to co-opt growth factor signaling pathways to counteract the activity of chemotherapy. We previously demonstrated that IGF-1 and heregulin, the ligands for IGF-1R and ErbB3 respectively, can decrease the cytotoxic activity of gemcitabine and nab-paclitaxel (Pace et al, Journal of Clinical Oncology, Volume 33, No. 3_suppl: A289; 2015). Additionally, co-expression of IGF-1R and ErbB3 is known to be associated with poor survival prognosis in MPC patients (Wang-Gillam et al, Journal of Clinical Oncology, Volume 33, No. 3_suppl: A295; 2015). Istiratumab (MM-141), a fully human bispecific antibody directed at IGF-1R and ErbB3, produced significant tumor regression, including durable complete responses, when combined with nab-paclitaxel and gemcitabine in preclinical models of pancreatic cancer, and has been shown to be safe and tolerable in a Phase 1 study (Isakoff et al, Journal of Clinical Oncology, Volume 33, No. 3_suppl: A384; 2015). On these bases, the current Phase 2 study was developed to evaluate istiratumab plus gemcitabine and nab-paclitaxel in MPC patients.

Cancer therapy treatment has advanced with the use of targeted agents that have significantly increased the utility of traditional chemotherapies as part of combination regimens. Most of the successes have been observed in those cancer subtypes in which a specific oncogenic protein is mutated, such as EGF receptor (EGFR), BRAF, or ALK, or the expression is amplified, such as ErbB2 in breast and gastric cancer. However, many patients never respond to these combination regimens or become refractory, suggesting the existence of uncharacterized tumor survival mechanisms. Although inhibition of IGF-1R was expected to eliminate a key resistance mechanism to anticancer therapies, clinical results to date have been disappointing. It has previously been established that adaptive v-erb-b2 erythroblastic leukemia viral oncogene homolog 3 (ErbB3) signaling activated by its ligand heregulin is a key factor limiting the utility of anti-IGF-1R agents. A series of biomolecules have been invented that co-inhibit IGF-1R and ErbB3, including a bispecific tetravalent antibody, MM-141, having the USAN “istiratumab”. Istiratumab is a polyvalent bispecific antibody (PBA) that co-blocks IGF-1 and heregulin-induced signaling and induces degradation of receptor complexes containing IGF-1R and ErbB3, including their respective heterodimers with insulin receptor and with ErbB2. MM-141 is disclosed in U.S. Pat. No. 8,476,409, which also discloses a number of other novel PBAs that, like istiratumab, bind specifically to human IGF-1R and to human ErbB3 and are potent inhibitors of tumor cell proliferation and of signal transduction through their actions on either or (typically, as for istiratumab) both of IGF-1R and ErbB3. The invention of such co-inhibitory biomolecules has resulted in a need for new approaches to combination therapies for cancer. The present invention addresses these needs and provides other benefits.

### SUMMARY

Provided herein are compositions comprising, and methods for use of istiratumab. It has now been discovered that therapeutic co-administration to a subject of istiratumab with nab-paclitaxel (ABRAXANE®) and gemcitabine (GEMZAR®), exhibits therapeutic synergy.

Accordingly, provided are methods for the treatment of a pancreatic cancer in a human patient (a “subject”) wherein the methods comprise administering to the subject a therapeutically effective amount of each of istiratumab, nab-paclitaxel, and gemcitabine.

In a particular embodiment, the cancer is a metastatic pancreatic cancer and the istiratumab is administered intravenously bi-weekly at a fixed dose of 2.8 g (i.e., 2.8 grams IV q 2 weeks) in combination with gemcitabine and nab-paclitaxel.

In some embodiments, co-administration of the gemcitabine and nab-paclitaxel with the istiratumab has an additive or superadditive effect on suppressing tumor growth, as compared to administration of matched doses of the istiratumab alone, or compared to matched doses of the gemcitabine and nab-paclitaxel administered together without the istiratumab. The effect on suppressing tumor growth may be measured in a subject, or in a mouse xenograft model using BxPC-3, Caki-1, SK-ES-1, A549, NCl/ADR-RES, BT-474, DU145, or MCF7 cells.

In some embodiments the patient has a cancer that is refractory to one or more anti-cancer agents, e.g., gemcitabine or paclitaxel.

In one aspect, a patient has a cancer and is selected for treatment and treatment is ordered by a healthcare provider (e.g., a physician) with the istiratumab as herein described, only if the patient has a serum concentration (level) of free IGF-1 (i.e., IGF-1 in serum that is not bound to an IGF-1 binding protein) that is above the population median level of free IGF-1 for patients with that type of cancer. In one embodiment, the patient has a pancreatic cancer and has a serum level of free IGF-1 that is above the pancreatic cancer population median level of 0.235 ng/ml of free serum IGF-1. Alternately, the serum concentration of free IGF-1 is 1.25, 1.5, 1.75, 2, 2.25, 2.5, 2.75, 3, 3.5, 4, 4.5, 5, 5.5, or 6 times the lower limit of detection for a particular assay, i.e., the assay described in Example 33. Alternately, the patient is treated with istiratumab only if the patient's serum free IGF-1 level meets a cutoff determined for the same type and stage of cancer as the patient. In one embodiment, the cutoff is above the population median level (i.e., the median level in a population of cancer patients with the same type of cancer as the patient). In another embodiment, the cutoff is below the population median level. In one embodiment, the cutoff is about 15%, about 10%, or about 5% below the population median level.

Also provided is a kit comprising a container holding at least one dose of istiratumab in a pharmaceutically-acceptable carrier. The kit optionally further comprises instructions to a practitioner, wherein the instructions call for a dosage of 2.8 grams of istiratumab administered intravenously every two weeks to a patient who is receiving co-administration of gemcitabine and nab-paclitaxel. In one embodiment, the kit further includes one or more devices that facilitate intravenous administration of the istiratumab. In another embodiment, the kit further comprises either or both of at least one dose (e.g., 0.25 grams or more) of nab-paclitexel and at least one dose (e.g., 2.0 grams or more) of gemcitabine.

In certain aspects, provided herein are: a method for treating or ordering treatment of a pancreatic cancer, optionally a KRAS mutant pancreatic cancer, in a subject, the treatment comprising co-administration to the subject 2.8 grams of istiratumab Q2W, 1000 mg/m² of gemcitabine QW, and 125 mg/m² of nab-paclitaxel QW. In certain aspects, the administration of each of istiratumab, gemcitabine and nab-paclitaxel is intravenous administration.

In one embodiment, provided herein is a method for treating or ordering treatment of a pancreatic cancer, optionally a KRAS mutant pancreatic cancer, in a subject, the treatment comprising co-administration to the subject 2.8 grams of istiratumab every two weeks (Q2W), 1000 mg/m² of gemcitabine once a week (QW) for three weeks followed by one week off, and 125 mg/m² of nab-paclitaxel once a week (QW) for three weeks followed by one week off.

In another embodiment, provided herein is a method for treating or ordering treatment of a pancreatic cancer, optionally a KRAS mutant pancreatic cancer, in a subject, the treatment comprising a 4-week treatment cycle comprising co-administration to the subject 2.8 grams of istiratumab Q2W at week 1 and week 3, 1000 mg/m² of gemcitabine once a week (QW) at week 1, week 2, and week 3, followed by week 4 off, and 125 mg/m² of nab-paclitaxel once a week (QW) at week 1, week 2, and week 3, followed by week 4 off.

In another embodiment, provided herein is a method for treating or ordering treatment of a pancreatic cancer, optionally a KRAS mutant pancreatic cancer, in a subject, the treatment comprising a 28-day treatment cycle comprising co-administration to the subject 2.8 grams of istiratumab Q2W on day 1 and day 15, 1000 mg/m² of gemcitabine on day 1, day 8, and day 15, and 125 mg/m² of nab-paclitaxel on day 1, day 8, and day 15.

In another embodiment, provided herein is a method for treating or ordering treatment of a pancreatic cancer, optionally a KRAS mutant pancreatic cancer, in a subject, the treatment comprising co-administration to the subject 2.24 grams of istiratumab every two weeks (Q2W), 1000 mg/m² of gemcitabine once a week (QW) for three weeks followed by one week off, and 125 mg/m² of nab-paclitaxel once a week (QW) for three weeks followed by one week off.

In another embodiment, provided herein is a method for treating or ordering treatment of a pancreatic cancer, optionally a KRAS mutant pancreatic cancer, in a subject, the treatment comprising a 4-week treatment cycle comprising co-administration to the subject 2.24 grams of istiratumab Q2W at week 1 and week 3, 1000 mg/m² of gemcitabine once a week (QW) at week 1, week 2, and week 3, followed by week 4 off, and 125 mg/m² of nab-paclitaxel once a week (QW) at week 1, week 2, and week 3, followed by week 4 off.

In another embodiment, provided herein is a method for treating or ordering treatment of a pancreatic cancer, optionally a KRAS mutant pancreatic cancer, in a subject, the treatment comprising a 28-day treatment cycle comprising co-administration to the subject 2.24 grams of istiratumab Q2W on days 1 and 15, 1000 mg/m² of gemcitabine on day 1, day 8, and day 15, and 125 mg/m² of nab-paclitaxel on day 1, day 8, and day 15.

In another embodiment, provided herein is a method for treating or ordering treatment of a pancreatic cancer, optionally a KRAS mutant pancreatic cancer, in a subject, the treatment comprising co-administration to the subject 1.96 grams of istiratumab every two weeks (Q2W), 1000 mg/m² of gemcitabine once a week (QW) for three weeks followed by one week off, and 125 mg/m² of nab-paclitaxel once a week (QW) for three weeks followed by one week off.

In another embodiment, provided herein is a method for treating or ordering treatment of a pancreatic cancer, optionally a KRAS mutant pancreatic cancer, in a subject, the treatment comprising a 4-week treatment cycle comprising co-administration to the subject 1.96 grams of istiratumab Q2W at week 1 and week 3, 1000 mg/m² of gemcitabine once a week (QW) at week 1, week 2, and week 3, followed by week 4 off, and 125 mg/m² of nab-paclitaxel once a week (QW) at week 1, week 2, and week 3, followed by week 4 off.

In another embodiment, provided herein is a method for treating or ordering treatment of a pancreatic cancer, optionally a KRAS mutant pancreatic cancer, in a subject, the treatment comprising a 28-day treatment cycle comprising co-administration to the subject 1.96 grams of istiratumab Q2W on day 1 and day 15, 1000 mg/m² of gemcitabine on day 1, day 8, and day 15, and 125 mg/m² of nab-paclitaxel on day 1, day 8, and day 15.

In one embodiment, the pancreatic cancer in the methods described herein is metastatic pancreatic cancer. In another embodiment, the pancreatic cancer in the methods described herein is metastatic adenocarcinoma of the pancreas.

In other aspects, methods are provided for a healthcare provider (e.g., a physician, physician's assistant, or other practitioner with prescribing authority, i.e., a prescribing healthcare provider) to treat, or order treatment of (e.g., by an administering healthcare provider such as a nurse), a pancreatic cancer patient whose level of free serum IGF-1 has been measured, the method comprising the healthcare provider reviewing the measured level of free serum IGF-1, and, if the level is at least a threshold level (optionally at least 0.235 ng/mL), treating (or ordering treatment of) the patient with the method of claim 1, and, optionally, if the level is less than the threshold level (optionally less than 0.235 ng/mL), the patient is treated with a therapeutic regimen that does not comprise administration of istiratumab.

### BRIEF DESCRIPTION OF THE DRAWINGS

FIG. 1 shows an overview of the Phase II trial described in Example 1.

FIG. 2 shows that istiratumab potentiates the activity of chemotherapy in HPAF-II KRAS mutant pancreatic tumors as described in Pace et al., 2015 (supra).

FIG. 3 shows that chemotherapy treatment increases HRG mRNA expression in CFPAC-1 cells. HRG mRNA expression was quantified after 24 h treatment with 1 mM of gemcitabine or 0.1 mM of paclitaxel, and normalized to housekeeping gene expression. “Vehicle”=drug-free vehicle control; “Gem”=gemcitabine treated; “Pac”=paclitaxel treated.

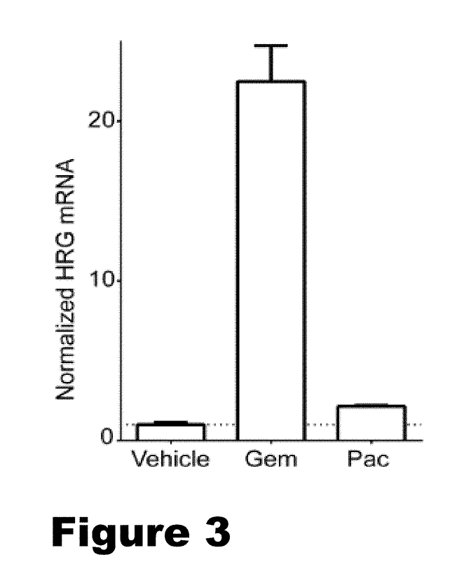

### DETAILED DESCRIPTION

### Methods and Compositions

Methods of combination therapy and combination compositions for treating cancer in a patient are provided. In these methods, the cancer patient is treated with istiratumab and nab-paclitaxel and gemcitabine.

The terms “combination therapy,” “co-administration,” “co-administered” or “concurrent administration” (or minor variations of these terms) include simultaneous administration of at least two therapeutic agents to a patient or their sequential administration within a time period during which the first administered therapeutic agent is still present in the patient (e.g., in the patient's plasma or serum) when the second administered therapeutic agent is administered.

The term “monotherapy” refers to administering a single drug to treat a disease or disorder in the absence of co-administration of any other therapeutic agent that is being administered to treat the same disease or disorder.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., as one or more administrations, e.g., either or both of orally (e.g., as one, two, three or more pills, capsules, etc.) or injected (e.g., as a bolus or infusion). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

“Effective amount” refers to an amount (administered in one or more doses) of an antibody, protein or additional therapeutic agent, which amount is sufficient to provide effective treatment.

“ErbB3” refers to ErbB3 protein, as described in U.S. Pat. No. 5,480,968. The human ErbB3 protein sequence is shown in SEQ ID NO:4 of U.S. Pat. No. 5,480,968, wherein the first 19 amino acids (aas) correspond to the leader sequence that is cleaved from the mature protein. ErbB3 is a member of the ErbB family of receptors, other members of which include ErbB1 (EGFR), ErbB2 (HER2/Neu) and ErbB4. ErbB3 itself lacks tyrosine kinase activity, but is itself phosphorylated upon dimerization of ErbB3 with another ErbB family receptor, e.g., ErbB1 (EGFR), ErbB2 and ErbB4, which are receptor tyrosine kinases. Ligands for the ErbB family receptors include heregulin (HRG), betacellulin (BTC), epidermal growth factor (EGF), heparin-binding epidermal growth factor (HB-EGF), transforming growth factor alpha (TGF-α), amphiregulin (AR), epigen (EPG) and epiregulin (EPR). The aa sequence of human ErbB3 is provided at Genbank Accession No. NP_001973.2 (receptor tyrosine-protein kinase erbB-3 isoform 1 precursor) and is assigned Gene ID: 2065.

“IGF-1R” or “IGF1R” refers to the receptor for insulin-like growth factor 1 (IGF-1, formerly known as somatomedin C). IGF-1R also binds to, and is activated by, insulin-like growth factor 2 (IGF-2). IGF1-R is a receptor tyrosine kinase, which, upon activation by IGF-1 or IGF-2, is auto-phosphorylated. The aa sequence of human IGF-1R precursor is provided at Genbank Accession No. NP_000866 and is assigned Gene ID: 3480.

Istiratumab (also known as “MM-141” and “P4-G1-M1.3”) is described as “P4-G1-M1.3” in WO/2012/145507 (PCT/US2012/034244), WO/2013/152034 (PCT/US2013/035013), WO/2015/130554 (PCT/US2015/016672), and U.S. Pat. No. 8,476,409, the teachings of all of which are expressly incorporated herein by reference. Istiratumab is a recombinant fully human bispecific tetravalent antibody. The complete tetrameric structure of the IgG1-based molecule is composed of two heavy chains (720 amino acids each) and two kappa light chains (214 amino acids each) held together by intrachain and inter-chain disulfide bonds. The variable regions of the heavy and light chains encode anti-IGF-1R modules. The C-terminus of the heavy chain encodes anti-ErbB3 scFv modules. MM-141-P5G5 is the designation for Master Cell Bank which produces MM-141. Istiratumab has two pairs of polypeptide chains, each pair of said two pairs comprising a heavy chain joined to a light chain by at least one heavy-light chain bond, wherein each light chain comprises the amino acid sequence set forth in SEQ ID NO: 1 and each heavy chain comprises the amino acid sequence set forth in SEQ ID NO: 2. SEQ ID NOs: 1 and 2 correspond to SEQ ID NOs: 204 and 226, respectively, as set forth in U.S. Pat. No. 8,476,409 (which is herein incorporated by reference in its entirety). In one embodiment, istiratumab comprises a linker having the amino acid sequence set forth in SEQ ID NO: 3. SEQ ID NO: 3 corresponds to SEQ ID NO: 53 as set forth in PCT/US2010/052712 (which is herein incorporated by reference in its entirety).

Istiratumab Formulation, Packaging, and Labeling:

Istiratumab is a colorless to slightly yellow liquid that may contain a low level of clear to white particles. Istiratumab will be supplied in sterile, single-use vials containing 47.6 mL of istiratumab (with extractable volume of 46.7 mL containing 280 mg of istiratumab) at a total protein concentration of 6.0 mg/mL in 20 mM histidine, 3% sucrose, 100 mM arginine-HCl, 0.005% Tween®80, pH 5.5. Istiratumab drug product should be stored at 2-8° C. The fill volume has been established to ensure an extractable volume of 46.7 mL (containing 280 mg of istiratumab). The appropriate volume of concentrate for solution for infusion is diluted into 0.9% saline solution for IV administration to the patient.

Gemcitabine (Gemzar®-CAS No. 122111-03-9) is indicated as a first line therapy for pancreatic adenocarcinoma and is also used in various combinations to treat ovarian, breast and non-small-cell lung cancers. Gemcitabine HCl is 2′-deoxy-2′,2′-difluorocytidine monohydrochloride (-isomer) (MW=299.66) and is administered parenterally, typically by i.v. infusion.

Paclitaxel (Taxol®-CAS No. 33069-62-4) is an anti-mitotic chemotherapy used for the treatment of lung, ovarian, breast and head and neck cancers.

Nab-paclitaxel (Abraxane®) is a nanoparticulate albumin-bound formulation of paclitaxel.

As used herein, “Q2W” refers to administration once every two weeks.

As used herein, “QW” refers to administration once a week.

### Outcomes

“Therapeutic synergy” refers to a phenomenon where treatment of patients with a combination of therapeutic agents manifests a therapeutically superior outcome to the outcome achieved by each individual constituent of the combination used at its optimum dose (T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

Thus, in combination, the components of such combinations have an additive or superadditive effect on suppressing tumor growth, as compared to monotherapy, e.g., with istiratumab or with gemcitabine or with nab-paclitaxel, or with one or two of these three compared to the triple combination. By “additive” is meant a result that is greater in extent (e.g., in the degree of reduction of tumor mitotic index or of tumor growth or in the degree of tumor shrinkage or the frequency and/or duration of symptom-free or symptom-reduced periods) than the best separate result achieved by monotherapy with each individual component, while “superadditive” is used to indicate a result that exceeds in extent the sum of such separate results. In one embodiment, the additive effect is measured as slowing or stopping of tumor growth. The additive effect can also be measured as, e.g., reduction in size of a tumor, reduction of tumor mitotic index, reduction in number of metastatic lesions over time, increase in overall response rate, or increase in median or overall survival.

### Kits and Unit Dosage Forms

Further provided are kits that include a pharmaceutical composition containing istiratumab, including a pharmaceutically-acceptable carrier, in a therapeutically effective amount adapted for use in the preceding methods. The kits include instructions to allow a practitioner (e.g., a physician, nurse, or physician's assistant) to administer the composition contained therein to treat an ErbB2 expressing cancer.

Optionally, instruments or devices necessary for administering the pharmaceutical composition(s) may be included in the kits. For instance, a kit may provide one or more pre-filled syringes containing an amount of istiratumab that allows for convenient delivery of 2.8 grams of istiratumab to a patient in a single dose.

Furthermore, the kits may also include additional components such as instructions or administration schedules for a patient suffering from a cancer to use the pharmaceutical composition(s) containing istiratumab.

### Rationale for Istiratumab in Pancreatic Cancer

Preclinical research indicates that:
IGF-1R and ErbB3 signaling are active in pancreatic cancer models.
Istiratumab inhibits growth factor-induced survival signaling.
Istiratumab reverses IGF-1- and HRG-mediated insensitivity to gemcitabine or paclitaxel.
Istiratumab potentiates the anti-tumor activity of nab-paclitaxel/gemcitabine in KRAS mutant pancreatic cancer models.

Negative clinical results using monospecific IGF-1R inhibitors in various cancers, including pancreatic, may be due to factors such as activation of compensatory signaling pathways that mediate resistance to treatment, as well as lack of biomarker-enriched patient selection.

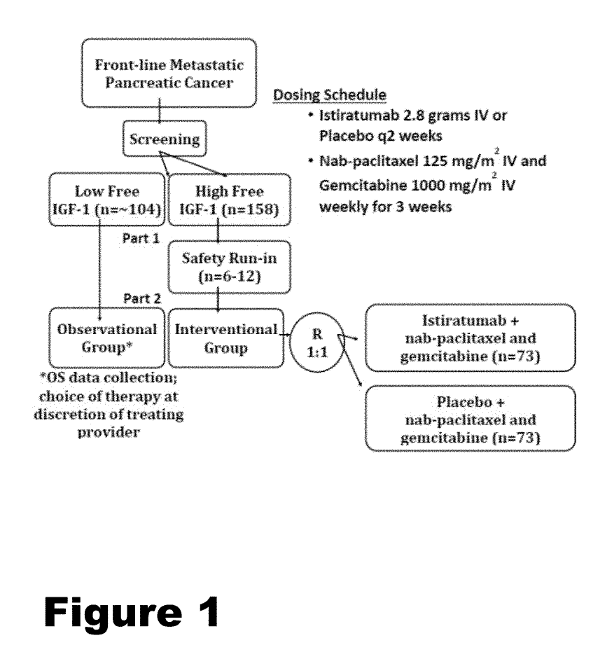

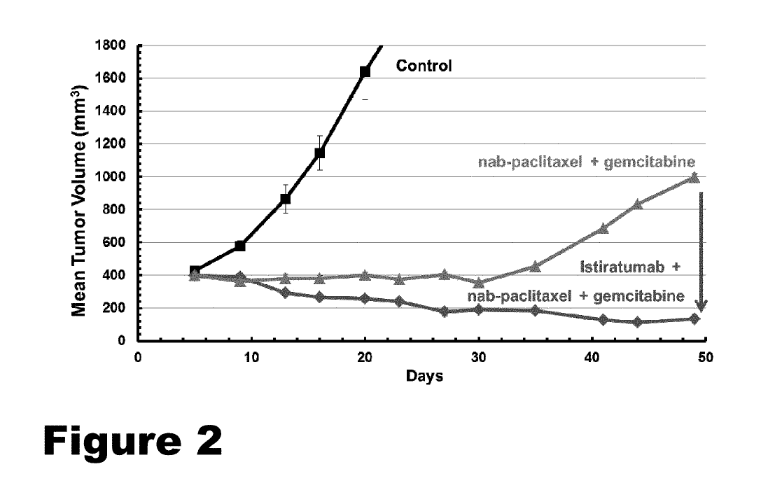

FIG. 1 shows that chemotherapy treatment increases HRG mRNA expression in CFPAC-1 cells and FIG. 2 shows that istiratumab potentiates the activity of chemotherapy in HPAF-II KRAS mutant pancreatic tumors.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The following Examples should not be construed as limiting the scope of this disclosure.

### Example 1: Phase 2 Trial

This Example provides details of a Phase 2 clinical trial of istiratumab in combination with gemcitabine and nab-paclitaxel for the treatment of MPC.

Methods:

Eligible patients have biopsy-confirmed MPC, ECOG PS 0-1, no prior therapy for advanced disease, and elevated serum levels of free IGF-1 (estimated to be ˜60% of all screened patients). The study design includes two parts. Part 1 (n=12) is an open-label assessment of the safety, tolerability, and pharmacokinetics (PK) of a fixed dose of istiratumab (2.8 grams IV Q2W) in combination with gemcitabine once a week (QW) for 3 weeks followed by one week off and nab-paclitaxel once a week (QW) for 3 weeks followed by one week off. In Part 2 (n=146), patients are randomized 1:1 and receive gemcitabine and nab-paclitaxel plus either istiratumab or placebo. The primary objective of Part 2 is to compare progression free survival (PFS) between the two treatment arms in two distinct cohorts: a) patients with high free serum IGF-1 levels (i.e., the entire study cohort), and b) patients with both high free serum IGF-1 levels and positive expression of heregulin in tumor tissue. Additional objectives include safety, overall survival, PK, and immunogenicity analyses of istiratumab, and correlative analyses of pre-defined biomarkers and their potential correlation with study outcomes. The study is designed with 80% power to detect a hazard ratio of 0.63 in favor of the experimental arm (8 vs 5 months) at a one-sided significance level of 0.05.

Patient serum for free serum IGF-1 level analysis is prepared by drawing whole blood into red top tubes, clotting 45-60 minutes at 2-8° C., and spinning down in a refrigerated centrifuge.

A preferred collection and preparation protocol is:
Draw blood into labelled “red-cap” tube.
Immediately after sample is drawn, gently invert the tube 180° and back, 5-6 times.
Allow serum samples to clot completely for 45-60 minutes at 2-8° C. in a vertical position. Observe that a dense clot has formed.
Keep tubes stoppered at all times.
Immediately after clotting occurs, centrifuge at 2-8° C. between 1100 and 1300 xg for 10 minutes for a swinging-head centrifuge or for 15 minutes for a fixed angle centrifuge.
Samples must be kept cold (2-8° C.) throughout the collection process.
Immediately after centrifugation, transfer serum to a storage tube. Pipette serum evenly into FOUR 3 mL “Cryovials.”
   Two Cryovials for “Primary” samples
   Two Cryovials for “Back-up” samples
Leave+20% of dead space in Cryovials to avoid cracking upon freezing.
Tightly stopper the Cryovials immediately.
Freeze immediately at <−80° C.
Store samples tightly stoppered at −80° C.±10° C. or below.
Samples must not undergo any freeze-thaw cycles.

Analysis of total IGF-1 in serum is performed by enzyme-linked immunosorbent assay (ELISA), e.g., using Human IGF-I Quantikine® ELISA Kit (R&D Systems, Minneapolis, Minn.) according to the manufacturer's instructions.

### Study Design and Objectives:

Study Design
Prospectively selected, randomized, double-blind, placebo-controlled, international multi-center Phase II trial. A study overview is provided in FIG. 1. Study treatment is administered as follows:

TABLE 1

| | Full Dose | Days of Administration |
|---|---|---|
| Drug* | Administration^{§} | within the 28 Day Cycle |
| Nab-paclitaxel | 125 mg/m² IV^{#} over | 1, 8, 15 |
| | 30-40 min | |
| Gemcitabine | 1000 mg/m² IV^{¥} over | 1, 8, 15 |
| | 30 min | |
| Istiratumab/ | 2.8 grams IV over | 1, 15 |
| Placebo | 90-120 min** | |

*Nab-paclitaxel is administered first, immediately followed by gemcitabine, then istiratumab/placebo. Premedication may be administered before istiratumab infusions. If steroids are given alone, they may be administered either before the chemotherapy infusions or before the istiratumab infusion. If triple premedication for istiratumab is indicated, the premedication agents may be given closer to the istiratumab infusion.
^{#}Recommended administration time of 30-40 min for nab-paclitaxel.
^{¥}Recommended administration time of 30 min for gemcitabine.
**Premedication with 10 mg of dexamethasone and infuse over 120 min with first dose in Cycle 1. If administration of istiratumab over 120 min is tolerated, the dose administration time of istiratumab/Placebo can be decreased to 90 min.

Istiratumab Dosage, Preparation, and Administration

Istiratumab is administrated as an IV infusion at 2.8 grams, which is 10 vials, every two weeks. Administration of istiratumab will require multiple vials and each administration should come from the same lot number. The first infusion of istiratumab or placebo is administered over 120 minutes, and subsequent infusions can be reduced to 90 minutes if the infusion is well tolerated. All infusions start after premedication has been administered.

Istiratumab is brought to room temperature prior to administration. For administration, the appropriate quantity of istiratumab is removed from the vial and injected into an empty 500 mL infusion bag. For placebo preparation, the appropriate quantity of normal saline is removed from the saline bag to match the infusion volume of an equivalent istiratumab dose. The infusion bag for both istiratumab and placebo is blinded by covering the bag with the provided IV bag cover. Study drug (either istiratumab or placebo) should be administered using a low protein binding 0.22 micrometer in-line filter. The line should be flushed before and after the study drug infusion. Study drug (istiratumab or placebo) should not be administered as a bolus or a push.

Primary Objectives
Determine if istiratumab plus nab-paclitaxel and gemcitabine is more effective than nab-paclitaxel and gemcitabine alone based on progression free survival (PFS) in:
   Serum free IGF-1 high population
   Serum free IGF-1 high and HRG positive tumor population

Key Secondary Objectives
Evaluate overall survival (OS), overall response rate (ORR), and duration of response (DoR) according to RECIST v 1.1
Describe the safety and tolerability of istiratumab plus nab-paclitaxel and gemcitabine
Compare OS in patients whose tumors are positive for heregulin

Key Exploratory Objectives
Evaluate OS in patients in the Observational Group
Describe the PK profile of istiratumab in patients with high free IGF-1 levels
Describe changes in CA19-9 and its potential correlation with outcome
Evaluate pre-defined biomarkers and their potential correlation with outcome

### Key Study Inclusion/Exclusion Criteria:

### Inclusion Criteria

Metastatic adenocarcinoma of the pancreas
No prior surgery, chemotherapy, or investigational therapy for the treatment of metastatic disease. Prior systemic treatment in the adjuvant setting (either alone and/or as a radiosensitizer) is only allowed if administered more than 6 months prior to enrollment in the study. Prior radiotherapy to metastatic lesions is considered on a case by case basis.
High serum levels of free IGF-1
ECOG performance status (PS) of 0 or 1
Adequate bone marrow reserve and renal function
Serum/plasma albumin levels >3 g/dL, total bilirubin within normal range
AST and ALT ≦2.5×ULN (≦5×ULN is acceptable if liver metastases are present)
Available recent tumor specimen or disease amenable to biopsy (Part 2 only)

### Exclusion Criteria

Patients who present with localized disease
Patients who are pregnant or lactating
Presence of an active infection
Patients with CNS malignancies or history of any malignancy in the last 3 years
Known hypersensitivity to the components of istiratumab, or who have had prior Grade 3-4 hypersensitivity reactions to human monoclonal antibodies
Prior treatment with any kind of IGF-1R or ErbB3 targeting agents
Known history of allergy or hypersensitivity to polysorbate (Tween) 80, arginine, nab-paclitaxel, gemcitabine, or their excipients
Clinically significant cardiac disease
Any episode of uncontrolled bleeding within the last 4 months
History of connective tissue disorders; interstitial lung disease, slowly progressive dyspnea and unproductive cough, sarcoidosis, silicosis, idiopathic pulmonary fibrosis, or pulmonary hypersensitivity pneumonitis; or peripheral artery disease
Use of string CYP3A4 and/or CYP2C8 inhibitors or inducers

### Current Status of the Phase II Study:

Part 1 of the study evaluating the safety, tolerability and pharmacokinetics of the 2.8 g fixed dose of istiratumab in combination with nab-paclitaxel+gemcitabine in MPC patients with high free IGF-1 in their serum completed enrollment. After completion of the safety, tolerability and PK analyses (approximately 6-8 weeks), the study re-opens to patient enrollment.

### Istiratumab/Placebo Pre-Medication and Management of Infusion-Related Reactions

Infusion reactions are defined according to the National Cancer Institute CTCAE (Version 4.0) definition of an allergic reaction/hypersensitivity. Infusion-related reactions are being considered an Adverse of Special Interest (AESI) on this study.

In order to prevent and manage infusion related reactions (IRRs), all patients should be pre-treated using the following guidelines prior to receiving infusions of istiratumab/placebo. Institutional guidelines should be followed if they differ from guidelines below.

TABLE 2
Premedication Schedule and Management
of Infusion-Related Reactions

| | | | Istiratumab/ | |
|---|---|---|---|---|
| | | | Placebo | |
| | | Treatment | Infusion | Subsequent |
| | Dose | (PO or IV) | Rate | Infusions |
| All | 1^{st} Dose | Pre-medicate | 120 minutes | See below |
| patients | | with | | |
| | | Dexametha- | | |
| | | sone | | |
| | | 10 mg | | |
| Infusion | | | | |
| Reaction | | | | |
| No | All | Pre-medicate | 90 minutes | No changes |
| reaction | subsequent | with | | |
| after 1^{st} | doses | Dexametha- | | |
| dose or | | sone | | |
| Grade 1 | | 10 mg | | |
| with any | | | | |
| dose | | | | |
| Grade 2 | 1^{st} | Pause | Resume at 50% | Pre-medicate |
| | occurrence | infusion | reduced rate | with |
| | | and | once symptoms | (PO or IV): |
| | | administer: | resolve | Dexametha- |
| | | Dexametha- | | sone |
| | | sone | | 20 mg |
| | | 20 mg | | Acetamin- |
| | | Acetamin- | | ophen |
| | | ophen | | |
| | | 500-650 mg | | 500-650 mg |
| | | Diphen- | | Diphen- |
| | | hydramine | | hydramine |
| | | 25-50 mg | | 25-50 mg |
| | | Broncho- | | Infuse |
| | | dilators, | | istiratumab/ |
| | | as necessary | | placebo over |
| | | | | 120 min |
| | Any other | Pause | Resume at 50% | Continue to |
| | occurrence | infusion | reduced rate | pre-medicate |
| | | and | once symptoms | as noted in |
| | | administer: | resolve | Grade 2 above |
| | | Dexametha- | | and infuse |
| | | sone | | istiratumab/ |
| | | 20 mg | | placebo over |
| | | Acetamin- | | 180 min |
| | | ophen | | |
| | | 500-650 mg | | |
| | | Diphen- | | |
| | | hydramine | | |
| | | 25-50 mg | | |
| | | Broncho- | | |
| | | dilators, | | |
| | | as necessary | | |
| Grade 3 | 1^{st} | Stop infusion | Do not resume | Continue to |
| | Occurrence | and | treatment at | pre-medicate |
| | | administer: | this visit | as noted in |
| | | Dexametha- | | Grade 2 above |
| | | sone | | and infuse |
| | | 20 mg | | istiratumab/ |
| | | Acetamin- | | placebo over |
| | | ophen | | 180 min |
| | | 500-650 mg | | |
| | | Diphen- | | |
| | | hydramine | | |
| | | 25-50 mg | | |
| | | Broncho- | | |
| | | dilators, | | |
| | | as necessary | | |
| | 2^{nd} | Stop infusion | Do not resume | No subsequent |
| | Occurrence | and | treatment | infusions. |
| | | administer: | | Patient should |
| | | Dexametha- | | be removed |
| | | sone | | from |
| | | 20 mg | | treatment. |
| | | Acetamin- | | |
| | | ophen | | |
| | | 500-650 mg | | |
| | | Diphen- | | |
| | | hydramine | | |
| | | 25-50 mg | | |
| | | Broncho- | | |
| | | dilators, | | |
| | | as necessary | | |
| Grade 4 | 1^{st} | Stop infusion | Do not resume | No subsequent |
| | Occurrence | and | treatment | infusions. |
| | | administer: | | Patient should |
| | | Dexametha- | | be removed |
| | | sone | | from |
| | | 20 mg | | treatment. |
| | | Acetamin- | | |
| | | ophen | | |
| | | 650 | | |
| | | Diphen- | | |
| | | hydramine | | |
| | | 25-50 mg | | |
| | | Broncho- | | |
| | | dilators, | | |
| | | as necessary | | |

For all patients who experience an infusion reaction of Grade ≧3, blood for a cytokine profile, complement levels and serum tryptase levels determination will be taken within 2 hours from the reaction, or as soon as clinically possible. In addition, 48 to 72 hours after this collection, blood will be drawn for human anti-human antibodies (HAHA) and repeat serum tryptase levels. Patients experiencing a second Grade 3 hypersensitivity reaction to istiratumab/placebo despite the use of pre-medication and any patients experiencing a Grade 4 hypersensitivity reaction to istiratumab/placebo should not be re-challenged. Such reactions, such as hypotension requiring treatment, dyspnea requiring bronchodilators, angioedema or generalized urticaria, require immediate discontinuation of the drugs and aggressive symptomatic therapy.

### Istiratumab/Placebo Dose Modifications

In the event of Grade 1 or 2 toxicities that are related to istiratumab treatment, no dose modification is recommended.

For Grade 3-4 toxicities that are possibly, probably or definitely related to istiratumab treatment, and not infusion-related reactions, dosing can be held for up to 21 days to allow for recovery of toxicity to ≦Grade 2 or baseline. Subsequent dosing will start at the dose reduction level, as outlined in Table 1. Patients who require a dose reduction should not have their doses re-escalated during their participation on the treatment portion of the study. In the event a Grade 3-4 toxicity occurs, but subsequent events can be managed with prophylactic or supportive care (i.e., nausea, vomiting, diarrhea, etc.), a dose reduction may not be required. Continued treatment at a reduced dose of istiratumab should be in the best interest of the patient. If the toxicity has not resolved after holding treatment for 21 days or if a dose reduction below 1.96 grams every 2 weeks is required, the patient should be removed from treatment.

TABLE 3
Dose Level Reductions for Istiratumab/placebo

| | | |
|---|---|---|
| Dose Level | Dose | Frequency |

| | | | |
|---|---|---|---|
| Full dose | 2.8 | g (10 vials) | Q2 weeks |
| 1^{st} dose reduction | 2.24 | g (8 vials) | Q2 weeks |
| 2^{nd} dose reduction | 1.96 | g (7 vials) | Q2 weeks |

| | | |
|---|---|---|
| If additional dose | Discontinue | Discontinue |
| reductions required | | |

### Nab-Paclitaxel and Gemcitabine Dose Modifications

In the event of toxicities related to nab-paclitaxel and gemcitabine, dose level reductions may be made as described in Table 4.

TABLE 4
Dose Level Reductions for Nab-paclitaxel and Gemcitabine

| | Nab-paclitaxel | Gemcitabine |
|---|---|---|
| Dose Level | (mg/m²) | (mg/m²) |
| Full dose | 125 | 1000 |
| 1^{st} dose reduction | 100 | 800 |
| 2^{nd} dose reduction | 75 | 600 |
| If additional dose | Discontinue | Discontinue |
| reductions required | | |

### Example 2: Diagnostic Strategy for Istiratumab Treatment

Based on historical and pre-clinical data, it is hypothesized that patients with IGF-1 or heregulin-dependent tumors derive more benefit from treatment with istiratumab.
Clinical trial data indicate high serum free IGF-1 levels correlate with improved survival of patients treated with IGF-1R inhibitors.
Retrospective analysis of the Phase 1 study (NCT01733004) found that patients with detectable pretreatment levels of serum IGF-1 remained on study approximately twice as long as those with non-detectable levels (Isakoff et al, 2015, supra).
Patients are prospectively selected based on high, pre-treatment, serum free IGF-1 levels using a serum ELISA-based assay. The assay is specific for free IGF-1 and does not detect physiologically relevant concentrations of IGF-2, or IGF-1 bound to IGF-BPs.
Pre-treatment tumor samples are retrospectively evaluated for HRG expression using a RNA in situ-hybridization (ISH)-based assay, e.g., according to the protocol described in Example 1 of U.S. Ser. No. 14/965,301; WO 2015/100459, which is expressly incorporated herein by reference.
In order to establish the threshold for inclusion/exclusion, 155 intended use samples (serum from untreated metastatic pancreatic cancer patients) and samples from patients screened for participation in the 2.8 g fixed-dose cohort of Part 1 were profiled using the free IGF-1 assay. Based on the observation from previous clinical studies, the median value of free IGF-1 was identified. The cut-point was established to be 10% below this median in order to account for the variance (CV) and is twice the lower limit of assay. All values equal to or above this cut-off are scored as “high” for free IGF-1 and allow for inclusion into Part 1 and the interventional group of the study.

Prospective Selection
A serum free IGF-1 ELISA assay, optionally a CLIA certified ELISA assay, is performed, for example, according to the following protocol.

100 μl of IGF1R-His (e.g., from R&D Systems) at 4.0 μg/ml in 1×PBS is added to Pierce™ Reacti-bind™ 96-well plates (Thermo Fisher Scientific) and incubated for 12 to 26 hours at 2-8° C. on a plate shaker set to 500 rpm.

Using an automated plate washer, each well is aspirated before adding 400 μL of room temperature Wash Buffer, followed by a 30-second soak. This is repeated three (3) additional times for a total of 4 washes.

Plates are blocked by the addition of 300 μL of room temperature Pierce™ Protein-Free (PBS) Blocking Buffer to each well. Incubation occurs at room temperature for 1 hour±10 min. Plates are washed as described above.

100 μL of standards, controls and unknown samples are added in duplicate and incubated for 2 hours±20 min at 2-8° C. on a plate shaker set to 500 rpm. Plates are washed as described above.

100 μL of rabbit anti-human IGF-1 antibody (ABCAM) is added and incubated for 1 hour±10 min at room temperature on a plate shaker set to 500 rpm. Plates are washed as described above.

100 μL of anti-rabbit IgG HRP-linked antibody (e.g., from Cell Signaling Technology, Inc.) is added and incubated for 1 hour±10 min at room temperature on a plate shaker set to 500 rpm. Plates are washed as described above.

100 μL of room temperature TMB substrate (Cell Signaling Technology, Inc.) is added and incubated in the dark for 10-15 minutes at room temperature until a blue color develops.

The TMB substrate reaction is stopped by adding 100 μL of room temperature Stop Solution (Cell Signaling Technology, Inc.) into each well.

The absorbance is measured at 450 nm with a 540 nm wavelength correction and within 30 min after the addition of the Stop Solution.

The SoftMax® PRO Enterprise Edition (software 5.2 or higher, Molecular Devices) is utilized to generate a four-parameter logistic (4 PL) curve by plotting the mean OD for each standard on the y-axis against the nominal concentration on the x-axis. Final concentrations are determined from the standard curve by interpolation.

Retrospective Review
Tissue heregulin RNA-ISH assay, optionally a CLIA certified RNA-ISH assay.

### SUMMARY OF SEQUENCE LISTING

Light Chain of polyvalent bispecific antibody
P4-G1-M1.3 (MM-141)
SEQ ID NO: 1
DIQMTQSPSSLSASLGDRVTITCRASQGISSYLAWYQQKPGKAPKLLIYA
KSTLQSGVPSRFSGSGSGTDFTLTISSLQPEDSATYYCQQYWTFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy Chain of polyvalent bispecific antibody
P-G1-M1.3 (MM-141)
SEQ ID NO: 2
EVQLLQSGGGLVQPGGSLRLSCAASGFMFSRYPMHWVRQAPGKGLEWVGS
ISGSGGATPYADSVKGRFTISRDNSKNTLYLQMNSLRAEDTAVYYCAKDF
YQILTGNAFDYWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GGGGGSGGGGSGGGGSQVQLVQSGGGLVQPGGSLRLSCAASGFTFDDYAM
HWVRQAPGKGLEWVAGISWDSGSTGYADSVKGRFTISRDNAKNSLYLQMN
SLRAEDTALYYCARDLGAYQWVEGFDYWGQGTLVTVSSASTGGGGSGGGG
SGGGGSGGGGSSYELTQDPAVSVALGQTVRITCQGDSLRSYYASWYQQKP
GQAPVLVIYGKNNRPSGIPDRFSGSTSGNSASLTITGAQAEDEADYYCNS
RDSPGNQWVFGGGTKVTVLG
Linker
SEQ ID NO: 3
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGGGGGSGGGGSGGGGS

### EQUIVALENTS AND INCORPORATION BY REFERENCE

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure. The disclosure of each and every U.S. and foreign patent and pending patent application and publication referred to herein is specifically incorporated by reference herein in its entirety for all purposes.

1. A method for treating a pancreatic cancer in a human patient, the treatment comprising co-administration to the patient of 2.8 grams of istiratumab Q2W, 1000 mg/m² of gemcitabine QW for 3 weeks followed by one week off, and 125 mg/m² of nab-paclitaxel QW for 3 weeks followed by one week off.

2. A method for treating a pancreatic cancer in a human patient, the treatment comprising co-administering to the patient 2.24 grams of istiratumab Q2W, 1000 mg/m² of gemcitabine QW for 3 weeks followed by one week off, and 125 mg/m² of nab-paclitaxel QW for 3 weeks followed by one week off.

3. A method for treating a pancreatic cancer in a human patient, the treatment comprising co-administering to the patient 1.96 grams of istiratumab Q2W, 1000 mg/m² of gemcitabine QW for 3 weeks followed by one week off, and 125 mg/m² of nab-paclitaxel QW for 3 weeks followed by one week off.

4. The method of claim 1, wherein the treatment comprises a 4-week treatment cycle.

5. The method of claim 4, wherein istiratumab is administered at week 1 and week 3, gemcitabine is administered at weeks 1, 2, and 3, and nab-paclitaxel is administered at weeks 1, 2, and 3.

6. The method of claim 1, wherein the treatment comprises a 28-day treatment cycle.

7. The method of claim 6, wherein istiratumab is administered on day 1 and day 15, gemcitabine is administered on day 1, day 8, and day 15, and nab-paclitaxel is administered on day 1, day 8, and day 15.

8. The method of claim 1, wherein the co-administration of each of istiratumab, gemcitabine and nab-paclitaxel is by intravenous administration.

9. The method of claim 8, wherein the treatment is ordered by a prescribing healthcare provider for execution by one or more administrating healthcare providers.

10. The method of claim 9, wherein the one or more administrating healthcare providers comprise a nurse.

11. A method for a healthcare provider to treat a human pancreatic cancer patient whose level of free serum IGF-1 has been measured, the method comprising the healthcare provider reviewing the measured level of free serum IGF-1, and, if the level is at least a threshold level (optionally at least 0.235 ng/mL), treating the patient with the method of claim 1.

12. The method of claim 1, wherein the pancreatic cancer is metastatic pancreatic cancer.

13. The method of claim 1, wherein the pancreatic cancer is metastatic adenocarcinoma of the pancreas.
